(19) United States
(12) Patent Application Publication
(10) Pub. No.: US 2026/0318810 A1
(43) Pub. Date: Oct. 15, 2026

(54) METHODS OF SEALING EFFECT EVALUATION FOR DRILLING FLUID

(71) Applicants: CNPC USA Corporation, Houston, TX (US); Beijing Huamei, Inc., Beijing (CN); China National Petroleum Corporation, Beijing (CN)

(72) Inventors: Nancy Zhou, Katy, TX (US); Tim Dunne, Katy, TX (US); Meng Lu, Katy, TX (US); Steven Vaughan, Katy, TX (US); Jiaxiang Ren, Houston, TX (US)

(73) Assignees: CNPC USA Corporation, Houston, TX (US); Beijing Huamei, Inc., Beijing (CN); China National Petroleum Corporation, Beijing (CN)

(21) Appl. No.: 19/178,806

(22) Filed: Apr. 14, 2025

(51) Int. Cl. G01N 15/08; G01N 33/24

(52) U.S. Cl. CPC G01N 15/0826; G01N 33/24

(57) ABSTRACT

A method for testing the efficacy of sealing and plugging agents includes collecting a formation sample from an oil and gas formation, preparing a testing disk with the formation sample and a nonpermeable substance, such as a resin, assembling the testing disk in a Permeability Plugging Apparatus, pressurizing a fluid having a sealing and plugging agent at a constant injection pressure to flow the fluid through the testing disk, collecting and recording volumes of fluid effluent over time to determine a flow rate, and determining efficacy of the sealing and plugging agent based at least in part upon the flow rate through the formation sample.

### BACKGROUND

### 1. Field of Disclosure

Embodiments of the present disclosure relate to evaluating the sealing and plugging effect of agents used in drilling fluid, and particularly, embodiments relate to testing sealing and plugging agents using a shale resin disk in a Permeability Plugging Apparatus.

### 2. Description of Related Art

In the oil and gas industry, the evaluation of drilling fluids is helpful for determining fluid properties, which can, in turn, ensure both the stability and the efficiency of drilling operations. Drilling fluids, also known as drilling muds, serve multiple functions, including cooling and lubricating the drill bit, carrying cuttings to the surface, and maintaining wellbore stability. The effectiveness of these fluids is paramount, particularly when drilling through challenging formations such as shale. Traditional methods for evaluating drilling fluids often involve conducting various laboratory and field tests to assess properties such as rheology, viscosity, lubricity, and others. These tests may be designed to simulate downhole conditions and thus attempt to predict the performance of drilling fluids under high temperature and high pressure (HTHP) environments.

Commonly, a Permeability Plugging Apparatus (PPA) may be used in evaluation and filtration tests of loss circulation materials, which can simulate HTHP conditions and uses a ceramic disk as the filter media. However, the method of using a PPA to test sealing and plugging agents (SPAs) can face several limitations. For example, the ceramic disks used in PPA tests have pore throat diameters that are significantly larger than those found in some oil and gas formations. This discrepancy can lead to inaccurate predictions of fluid performance in actual downhole conditions, such as in shale formations. Additionally, conventional testing methods may not adequately simulate the complex interactions between drilling fluids and shale formations, which can result in drilling fluid formulations with suboptimal efficiency levels, as well as increased risks of wellbore instability.

### SUMMARY

Applicants recognized the problems noted above herein and conceived and developed embodiments of methods, according to the present disclosure, for evaluating sealing and plugging agents by testing permeability of shale samples within a shale resin disk, using a PPA.

In an embodiment, a method for testing efficacy of a sealing and plugging agent includes collecting a formation sample from an oil and gas formation, preparing a testing disk having the formation sample, where a portion of the testing disk has a nonpermeable substance, and assembling the testing disk in a PPA. The method may further include filling a cell of the PPA with a first fluid, pressurizing the first fluid at a first constant injection pressure, flowing the first fluid through the testing disk, where first fluid effluent flows out of the testing disk, collecting first fluid effluent, and recording volumes of the first fluid effluent over time to determine a first flow rate. Also, methods may include filling the cell of the PPA with a second fluid comprising the first fluid and a sealing and plugging agent (SPA), pressurizing the second fluid at a second constant injection pressure, flowing the second fluid through the testing disk, where second fluid effluent flows out of the testing disk, collecting the second fluid effluent, and recording volumes of the second fluid effluent over time to determine a second flow rate. Furthermore, the method may include determining an efficacy of the SPA based at least in part upon the first flow rate and the second flow rate.

In another embodiment, a method for testing and ranking efficacies of sealing and plugging agents includes preparing a first fluid with a first SPA, pressurizing the first fluid at a constant injection pressure and against a first formation sample of a first type, and recording volumes of first fluid effluent over time to determine a first flow rate, where the first fluid effluent flows from the first formation sample. The method may also include preparing a second fluid with a second SPA, pressurizing the second fluid at the constant injection pressure and against a second formation sample of the first type, and recording volumes of second fluid effluent over time to determine a second flow rate, where the second fluid effluent flows from the second formation sample. The method may further include ranking the first SPA relative to the second SPA based on the first flow rate and the second flow rate.

### BRIEF DESCRIPTION OF DRAWINGS

The present technology will be better understood on reading the following detailed description of non-limiting embodiments thereof, and on examining the accompanying drawings, in which:

FIGS. 1A and 1B are top perspective views of embodiments of a shale-resin disk used in testing sealing and plugging agents, in accordance with embodiments of the present disclosure;

FIG. 2 is a schematic diagram of a shale-resin disk inserted into a Permeability Plugging Apparatus used in testing sealing and plugging agents, in accordance with embodiments of the present disclosure;

FIG. 3 is a flow chart of a method for testing efficacy of a sealing and plugging agent, in accordance with embodiments of the present disclosure;

FIG. 4 is a flow chart of a method for testing and ranking sealing and plugging agents, in accordance with embodiments of the present disclosure; and

FIGS. 5A-5C are charts of fluid filtrate volume collected over time and pressure over time when testing and ranking sealing and plugging agents, in accordance with embodiments of the present disclosure.

### DETAILED DESCRIPTION

The foregoing aspects, features, and advantages of the present disclosure will be further appreciated when considered with reference to the following description of embodiments and accompanying drawings. In describing the embodiments of the disclosure illustrated in the appended drawings, specific terminology will be used for the sake of clarity. However, the disclosure is not intended to be limited to the specific terms used, and it is to be understood that each specific term includes equivalents that operate in a similar manner to accomplish a similar purpose. Additionally, like reference numerals may be used for like components, but such use should not be interpreted as limiting the disclosure.

When introducing elements of various embodiments of the present disclosure, the articles “a”, “an”, “the”, and “said” are intended to mean that there are one or more of the elements. The terms “comprising”, “including”, and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment”, “an embodiment”, “certain embodiments”, or “other embodiments” of the present disclosure are not intended to be interpreted as excluding the existence of additional embodiments that also incorporate the recited features. Furthermore, reference to terms such as “above”, “below”, “upper”, “lower”, “side”, “front”, “back”, or other terms regarding orientation or direction are made with reference to the illustrated embodiments and are not intended to be limiting or exclude other orientations or directions. Moreover, references to “substantially” or “approximately” or “about” may refer to differences within ranges of +/−10 percent. Furthermore, like numbers may be used to refer to like elements throughout, but it should be appreciated that using like numbers is for convenience and clarity and not intended to limit embodiments of the present disclosure. For example, one or more subsequent figures may share similar features with one or more prior figures and the similar features may be identified with like reference numerals for convenience purposes only and not to limit the scope of the present disclosure.

A shale-resin disk is prepared to be used in the PPA to study shale properties, such as, inter alia, water sensitivity, fracture pressure, and permeability of shale cuttings/chips. With the shale-resin disk, methods of the present disclosure include testing to evaluate the sealing and plugging effect of one or more SPAs that can be added to drilling fluid to prevent formation instability when carrying out oil and gas fracturing and extraction operations in sensitive oil and gas formations, such as shale formations.

A PPA has been used in the oil and gas industry to conduct evaluation and filtration tests of loss circulation materials. A PPA may be designed to simulate downhole HTHP static filtration. In a typical PPA, the filter media is a ceramic disk. API (American Petroleum Institute) establishes the designations of ceramic disks with 10 μm to 120 μm of mean pore throat diameter. Such diameters of the ceramic disks are much larger than the pore throat of a shale formation. Therefore, sealing effect evaluation using a PPA with a ceramic disk is not a representative manner of testing SPAs that are used for shale formation drilling. With the preparation of the shale resin-disk to replace the ceramic disk in the PPA, shale cuttings/chips directly from shale formation can be used as the filter media in the PPA. This arrangement may allow for better predictions on what will happen in the downhole application of the SPA in a drilling fluid, leading to more practical and reliable results.

In various embodiments, methods of testing the permeability of a shale sample may include applying reservoir brine or drilling fluid to a shale-resin disk. The fluid may be applied to the disk using a pump to control the injection rate and pressure. When SPAs are added into the fluid, methods may further include determining a sealing and plugging effect of the SPA for the shale sample by comparing the effluent collection rate (i.e., volumetric flow rate of effluent coming out of the shale-resin disk) and breakthrough pressure, if observed. Accordingly, a comparison of the sealing and plugging effect of various SPAs may be obtained, and a ranking may be formulated. The ranking of the SPAs may be based, at least in part, on the change in volumetric flow rate of the effluent leaving the shale-resin disk when the fluid did not include an SPA, to the volumetric flow rate of the effluent leaving the shale-resin disk when the fluid includes an SPA. Such a change in volumetric flow rate before and after adding an SPA may be indicative of the efficacy of the SPA in sealing and/or plugging the pores of the shale sample in the disk. In some scenarios, a sealing effect of an SPA is preferable. In other scenarios, a plugging effect of an SPA is preferable.

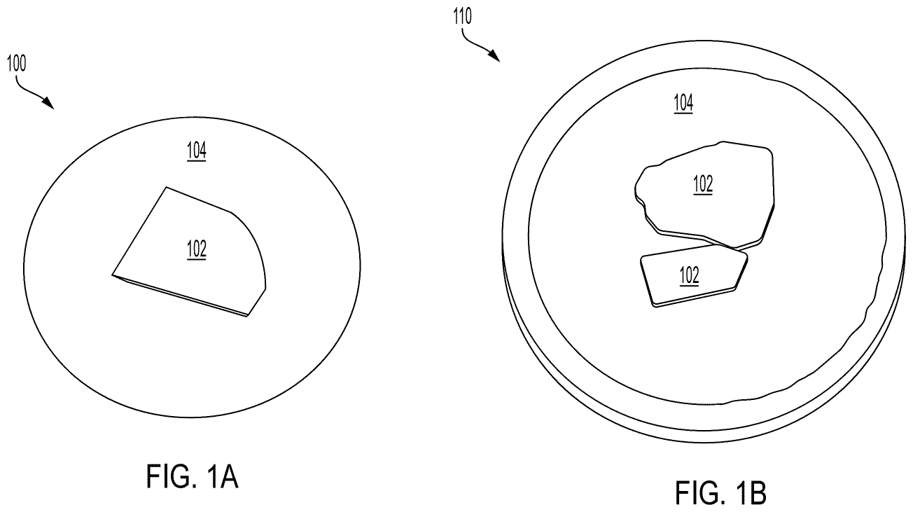

FIG. 1A is a top perspective view of an embodiment of a shale-resin disk 100 (e.g. a “testing disk”). The shale-resin disk 100 may include a formation sample 102 and a resin portion 104. The resin 104 may be nonpermeable and the formation sample 102 may be permeable. It should be appreciated that the disk 100 may not necessarily include a resin 104, and may instead include a different nonpermeable substance. The upper temperature ranges of the shale-resin disk 100 may be determined by resin chemistry, from approximately room temperature up to approximately 300 degrees Fahrenheit. It should be appreciated that different resins 104 may have different temperature ranges.

As illustrated in FIG. 1A, the shale-resin disk 100 includes one formation sample 102, however, there may be any reasonable number of formation samples 102 included in the shale-resin disk 100. For example, FIG. 1B shows a shale-resin disk 110 that includes two formation samples 102 within the resin 104. The formation sample 102 may be a sample from a shale formation, or a sample from another type of oil and gas formation. The type of formation sample 102 chosen for testing the efficacy of SPAs may depend on what type of formation that drilling and extraction operations are planned to occur.

As illustrated in FIGS. 1A and 1B, the one or more formation samples 102 are substantially encompassed and enveloped by the resin 104. Before using the disks 100, 110 in a PPA, the top and bottom surfaces (i.e., flat surfaces) of the disks 100, 110 may be ground using a grinding wheel, sandpaper, or other grinding or polishing equipment. The purpose of grinding the disks 100, 110 may be to at least partially remove the resin 104 on the top and bottom of the disks 100, 110 in order to expose the formation sample 102. Then, when fluid is applied to the shale-resin disks 100, 110, the fluid is able to contact the formation sample 102 on one side and given the opportunity to flow through the formation sample 102 to the other side of the disk 100, 110. It should be appreciated that the disks 100, 110 may also be punctured, drilled, or otherwise cut in a different manner to expose the formation sample 102 within the disk 100, 110 and perform permeability testing.

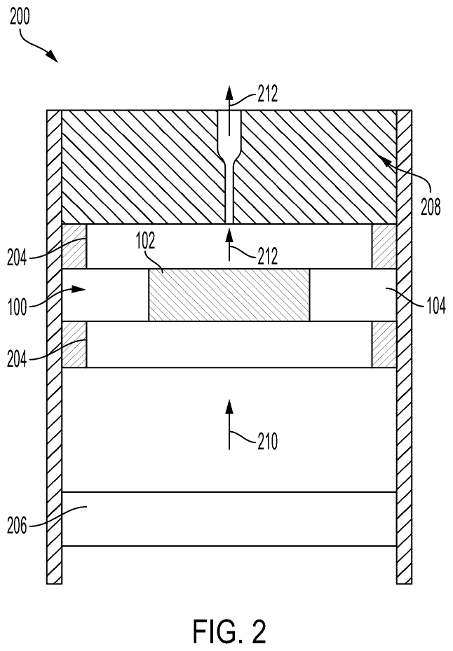

FIG. 2 is a schematic diagram of the shale-resin disk 100 inserted into a PPA 200. As described in FIGS. 1A and 1B, the shale-resin disk includes a formation sample 102 and a resin 104. In some embodiments, the PPA 200 further includes one or more O-rings 204 that may be included for the shale-resin disk 100 to rest between. The one or more O-rings 204 may ensure that fluid flowing through the PPA 200 is forced toward the formation sample 102 in the shale-resin disk 100 by sealing the edges of the shale-resin disc 100. The one or more O-rings 204 may also be any other type of sealing piece to ensure there is no unwanted fluid leaking or seeping. Within the PPA 200, there may be a floating piston body 206 and a top cap 208. The floating piston body 206 may serve the purpose of pushing the injected fluid 210 in the PPA cell toward the shale-resin disk 100. The PPA cell may also fill with fluid in the PPA 200. The top cap 208 may serve the purpose of directing any effluent fluid 212 out of the shale-resin disk 100 and out of the PPA 200 into a container for collection and measurement (container not illustrated in FIG. 2). As can be seen in FIG. 2, injected fluid 210 in the PPA cell flows upwards until it makes contact with the first O-ring 204 and the shale-resin disk 100. If the injected fluid 210 flows through the formation sample 102 of the shale-resin disk 100, the effluent fluid 212 passes the second O-ring 204 and continues upward in the PPA 200, through the top cap 208, and out of the PPA 200.

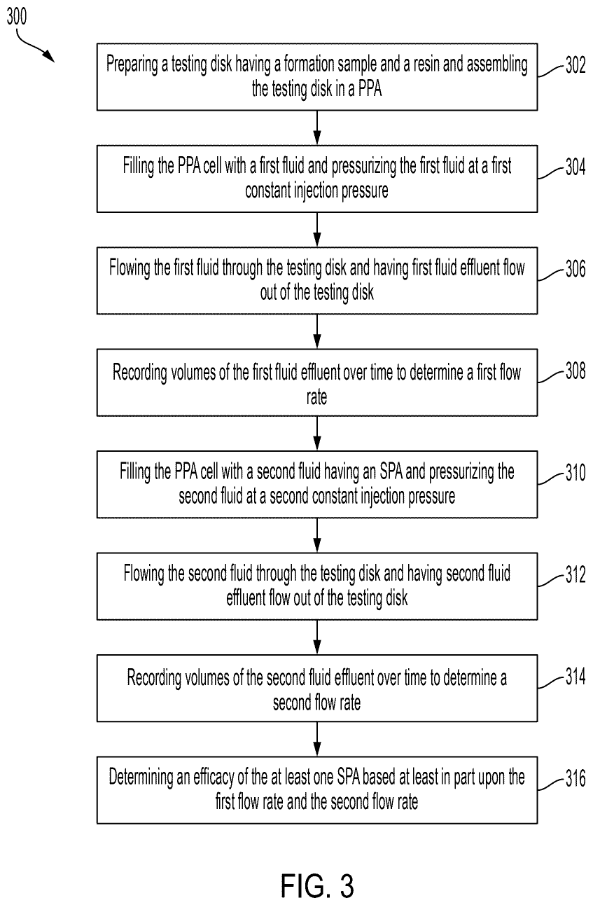

FIG. 3 is a flow chart of a method for testing efficacy of an SPA 300. The method 300 may include preparing a testing disk having a formation sample and a nonpermeable substance, such as a resin, and assembling the testing disk in a PPA 302. The method 300 also includes filling a cell of a PPA with a first fluid and pressurizing the first fluid at a first constant injection pressure 304. In various embodiments, the first fluid is potassium chloride (KCl) solution, a sodium chloride (NaCl) solution, another salt solution, water, and others. In addition, the first constant injection pressure may be approximately 1 psi to 100 psi, however, the injection pressure may be more, depending at least in part on the type of the formation sample, the width of the formation sample, the permeability of the formation sample, the type of SPA used, or other factors. The upper pressure of the test is up to 6000 psi of PPA.

The method for testing efficacy of an SPA 300 may further include flowing the first fluid through the testing disk and having first fluid effluent (or, first fluid filtrate) flow out of the testing disk 306. Methods may also include collecting the first fluid effluent in a container and recording volumes of the first fluid effluent over time to determine a first flow rate of the first fluid effluent flowing through the formation sample 308. In some embodiments, the first fluid effluent may be collected in a beaker or pipette having volumetric markings. In other embodiments, a flow meter to automatically measure velocity of the fluid effluent may be used.

The method 300 may further include filling the cell of the PPA with a second fluid and pressurizing the second fluid at a second constant injection pressure 310. In various embodiments, the second fluid includes an SPA along with the fluid used for the first fluid. The SPA may be nano-size carbon black. The SPA may also be NZ76-25A, which may be a nano size polymer from monomer of Acrylamide, and/or 2-Acrylamido-2-methyl 1-Propanesulfonic acid, and/or Diallyldimethylammonium chloride. Further, the SPA may be NZ76-25B, which may be hybrid nano- to micro-size particles from inorganic and organic chemicals, with a surfactant for dispersion of the particles in water. The SPA may also be NZ76-25C, which may be carbon black. The SPA may further be NZ76-25F, which may be a white powder of synthesis polymer. It should be appreciated that the SPA selected may also be any combination of the foregoing materials, along with other recognized SPAs, including nano-size carbon black or combinations thereof. Similar to what may be done with the first fluid described above, the method 300 may also include flowing the second fluid through the testing disk and having second fluid effluent/filtrate flow out of the testing disk 312, as well as recording volumes of the second fluid effluent over time to determine a second flow rate 314. Furthermore, the method 300 may include determining an efficacy of the SPA based at least in part upon the first flow rate and the second flow rate 316.

In some embodiments, the first constant injection pressure may be lower than the second constant injection pressure. In such embodiments, the first flow rate at the first, lower constant injection pressure is higher than the second flow rate at the second, higher constant injection pressure. Such a scenario may indicate a sealing or plugging effect of the SPA used in the second fluid, as flow rate is lower despite the higher injection pressure. In other embodiments of the first constant injection pressure being lower than the second constant injection pressure, the first flow rate is lower than the second flow rate. Accordingly, the first flow rate at the first, lower constant injection pressure is lower than the second flow rate at the second, higher constant injection pressure. Such a scenario may indicate that fractures have formed in the formation sample due to, inter alia, the higher injection pressure of the fluid.

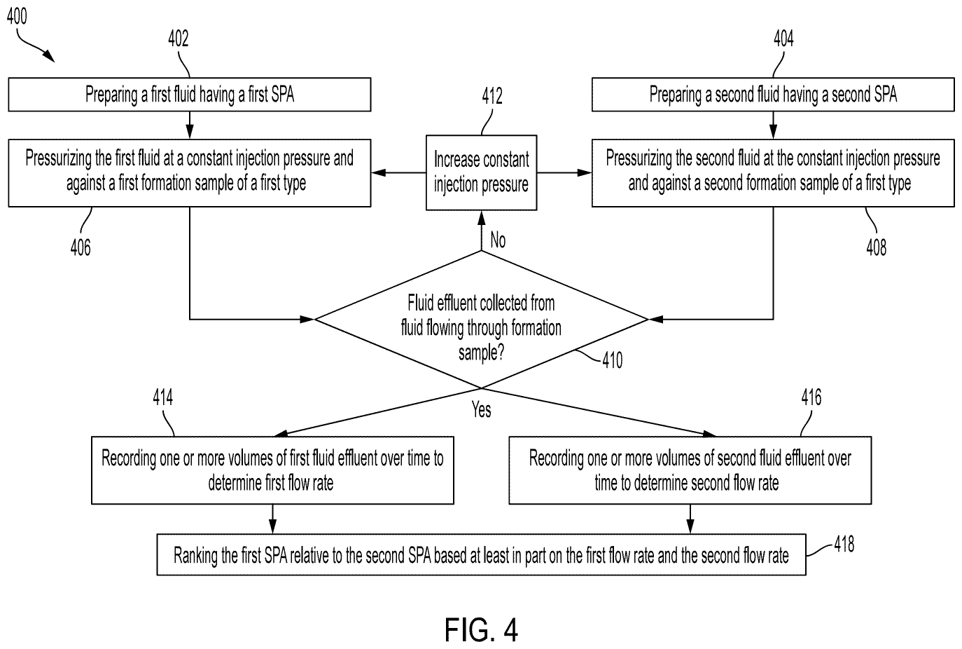

FIG. 4 is a flow chart of a method for testing and ranking sealing and plugging agents 400. The method 400 may include preparing a first fluid comprising a first SPA 402 and preparing a second fluid comprising a second SPA 404. It should be appreciated that more than two fluids with more than two SPAs may be prepared, and two fluids with two SPAs is given purely as an example and is not meant to limit the scope of the present disclosure. It should also be appreciated that the method 400 may or may not be carried out in a PPA using one or more testing disks, such as one or more shale-resin disks. The first fluid may be pressurized at a constant injection pressure and then injected or otherwise flowed towards or against a first formation sample of a first type 406. Similarly, the second fluid may be pressurized at that same constant injected pressure and then injected or otherwise flowed towards or against a second formation sample of that same first type 408. The first type of formation samples may be shale formation samples and may have been collected from the same oil and gas formation.

The method 400 may further include determining whether the first fluid and whether the second fluid has flowed through the formation sample to create respective first fluid effluent and second fluid effluent 410. If the first fluid has not flowed through the formation sample to create first fluid effluent or if the second fluid has not flowed through the formation sample to create second fluid effluent, then the constant injection pressure may be increased to a higher constant injection pressure 412. Subsequently, the first and second fluid would then be pressurized at the higher constant injection pressure and injected or otherwise flowed towards or against the respective first and second formation samples of the first type 406, 408. If the first and second fluid have flowed through the formation sample to create first and second fluid effluent, then the method may include recording one or more volumes of first fluid effluent over time to determine first flow rate 414 and recording one or more volumes of second fluid effluent over time to determine second flow rate 416. Furthermore, the method 400 may include ranking the first SPA relative to the second SPA based, at least in part, on the first flow rate and the second flow rate 418. The ranking may also be based, at least in part, on other factors, such as whether or not fractures occurred in the first formation sample and/or the second formation sample.

### EXAMPLES

The following examples are exemplary embodiments and are not meant to limit the scope of the disclosure.

### Example 1

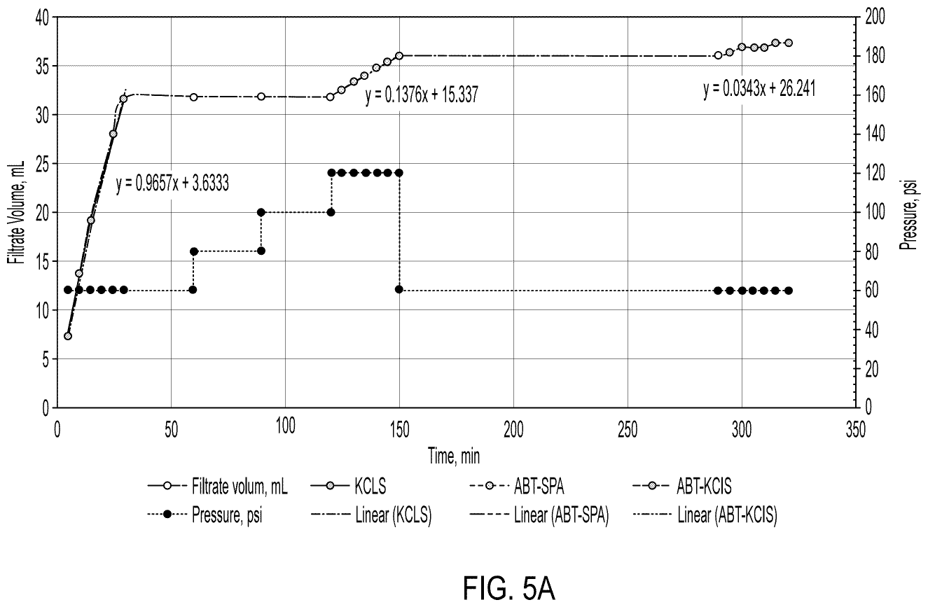

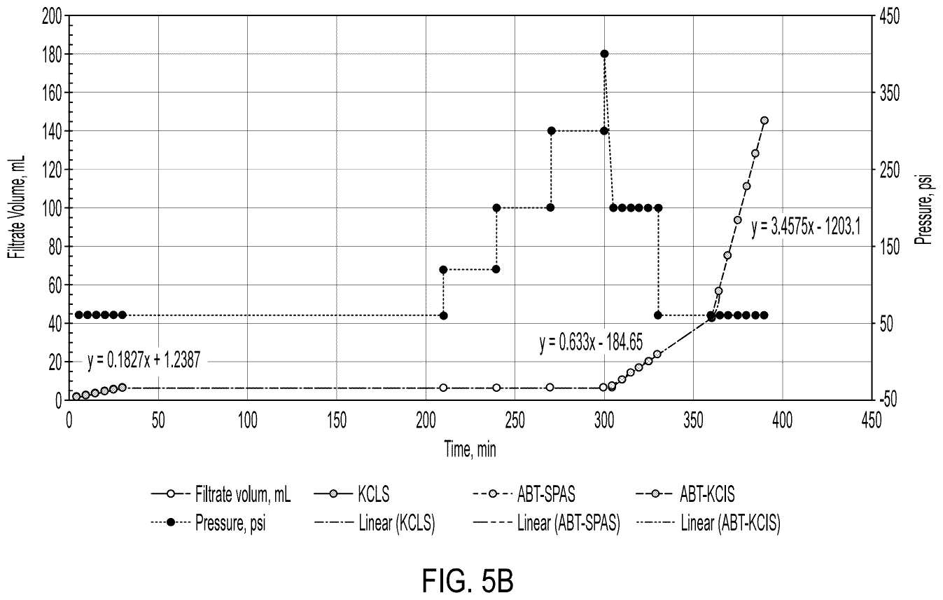

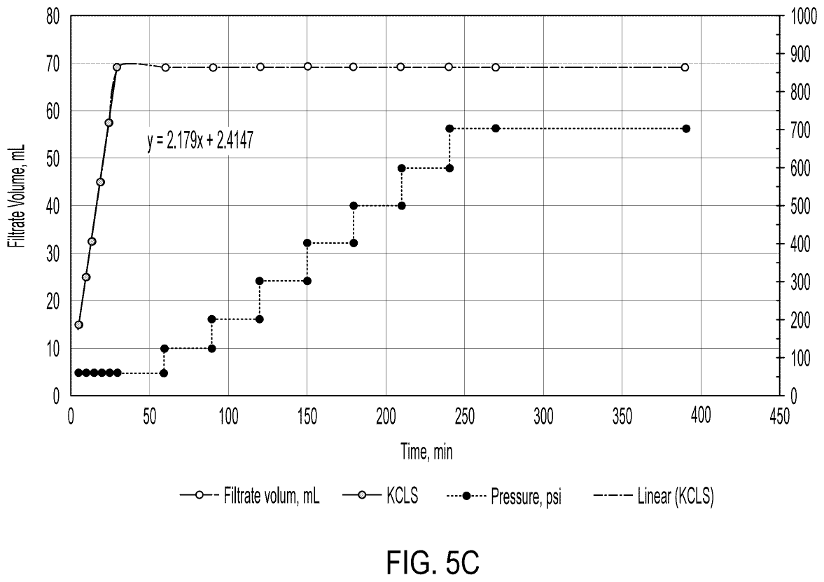

An experiment was conducted to develop an evaluation method to compare the efficacies of SPAs or otherwise screen SPAs. A shale-resin disk with a sample of shale (e.g., “TM-1”) was prepared then saturated in 10% KCl for 30 min. After that, PPA cell was assembled with KCl solution in the cell body. The pump was turned on to flow the KCl solution and an injection pressure of 60 psi was first selected. The KCl solution flowed through the shale-resin disk and various volumes of effluent filtrate from the disk were collected and recorded over time, as can be seen in FIG. 5A. In order to analyze the collected data, a trend line was fit to the data and an approximate volumetric flow rate was determined. For the KCl solution, the volumetric flow rate of the KCl solution effluent was approximately 0.96 mL/min.

Next, a fluid with SPA was prepared with a commercially available SPA: NZ76-25A. 80 g of NZ76-25A was diluted in 10% KCl solution to make a 480 g SPA solution. The PPA cell was disassembled and then the cell body was filled with the SPA solution. The pump was turned on to flow the SPA solution and an injection pressure of 60 psi was first selected. The injection pressure of 60 psi was sustained for 30 min and no effluent fluid was observed. Injection pressure was increased to 80 psi and sustained for another 30 min and there was also no effluent fluid observed. Injection pressure was again increased, now up to 100 psi, and again sustained for 30 min, and still no effluent fluid was observed. When injection pressure was increased to 120 psi, effluent fluid of the SPA solution was observed and various volumes of filtrate were collected and recorded over time, as can be seen in FIG. 5A. Again a trend line was fit to the data and an approximate volumetric flow rate was determined. For the SPA solution, the volumetric flow rate of the SPA solution effluent was approximately 0.14 mL/min.

The volumetric flow rate of the KCl solution with no SPA of approximately 0.96 mL/min indicated that the TM-1 shale needs a sealing and plugging agent to prevent or delay the instability of the corresponding wellbore location when pressure of a drilling fluid pumped downhole is over 60 psi. In this scenario, the change in volumetric flow rate, from approximately 0.96 mL/min for the KCl solution at an injection pressure of 60 psi, down to approximately 0.14 mL/min for the SPA solution at an injection pressure of 120 psi, demonstrated that additive NZ 76-25A in the SPA solution exhibits a sealing effect, such that fluid flow is restricted, at least partially, through the shale sample in the shale-resin disk.

### Example 2

A shale-resin disk with a sample of shale was prepared and then saturated in 10% KCl for 30 min. The effluent flow rate during the injection of the KCl solution through the shale-resin disk was approximately 0.18 mL/min, as indicated in FIG. 5B (the slope of the leftmost filtrate volume line in the figure). After that, a fluid with an SPA, NZ76-25F, was injected into the shale-resin disk of the PPA at 60 psi and no fluid effluent was recorded for 180 min, which was interpreted as no measurable fluid infusion or no measurable permeability of the shale sample. Therefore, the injection pressure was increased step by step to induce fractures, and the fractures may lead to fluid infusion when the fractures connect. Once fractures occur in the shale sample, then the effluent fluid volume may be observed over time. The injection pressure increment was set at 60 psi for 180 min, then 120 psi for 30 min, then 200 psi for 30 min, then 300 psi for 30 min, and lastly, up to 400 psi. At 400 psi, breakthrough was detected and fluid began flowing through the disk. This pressure is defined as the breakthrough pressure. The plugging effect of NZ76-25F is demonstrated by the flat line for filtrate volume in FIG. 5B until injection pressure was increased to 400 psi and effluent fluid began to be recorded finally. Also seen in FIG. 5B, the increasing slope changes of the filtrate volume line in the chart indicates that induced fractures occurred in the shale sample that caused a progressive increase of fluid flow through the sample.

### Example 3

A shale-resin disk with a sample of shale was prepared then saturated in a KCl solution for 30 min. The effluent flow rate during the injection of the KCl solution through the shale-resin disk was approximately 2.2 mL/min, as indicated in FIG. 5C. Then, fluid with NZ 76-25B, an SPA, was injected into the PPA using a pump, with the injection pressure being 60 psi initially. At 60 psi, no effluent flow was collected, which was interpreted as NZ76-25B plugging the pores of the shale sample sufficiently such that fluid could not flow through the shale-resin disk. After no effluent was collected after 30 minutes at 60 psi, injection pressure was increased to 100 psi for 30 minutes. There was still no effluent flow at 100 psi over these next 30 minutes. Subsequently, injection pressure was increased in increments of 100 psi, up to 200 psi, then 300 psi, and so on, up to 700 psi, each injection pressure being sustained for a period of 30 minutes. After approximately 5 total hours at pressures up to 700 psi, still no breakthrough fluid flow from the disk was observed. A graphical illustration of data collected during this Example 3 is displayed as FIG. 5C. As can be seen, filtrate volume is constant from 30 minutes to almost 400 minutes. This example demonstrates the strong plugging effect of this particular SPA, NZ76-25B.

Although the technology herein has been described with reference to particular embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

What is claimed is:

1. A method for testing efficacy of a sealing and plugging agent, comprising:
collecting a formation sample from an oil and gas formation;
preparing a testing disk comprising the formation sample, wherein a portion of the testing disk comprises a nonpermeable substance;
assembling the testing disk in a Permeability Plugging Apparatus (PPA);
filling a cell of the PPA with a first fluid;
pressurizing the first fluid at a first constant injection pressure;
flowing the first fluid through the testing disk, wherein first fluid effluent flows out of the testing disk;
collecting first fluid effluent;
recording one or more volumes of the first fluid effluent over time to determine a first flow rate;
filling the cell of the PPA with a second fluid comprising the first fluid and at least one sealing and plugging agent (SPA);
pressurizing the second fluid at a second constant injection pressure;
flowing the second fluid through the testing disk, wherein second fluid effluent flows out of the testing disk;
collecting the second fluid effluent;
recording one or more volumes of the second fluid effluent over time to determine a second flow rate; and
determining an efficacy of the at least one SPA based at least in part upon the first flow rate and the second flow rate.

2. The method of claim 1, wherein the first fluid is a potassium chloride solution.

3. The method of claim 1, wherein the at least one SPA comprises nano-size carbon black.

4. The method of claim 1, wherein the first constant injection pressure is lower than the second constant injection pressure.

5. The method of claim 4, wherein the first flow rate is higher than the second flow rate.

6. The method of claim 5, wherein the first flow rate being higher than the second flow rate indicates that the at least one SPA produces a sealing effect for the formation sample.

7. The method of claim 4, wherein the first flow rate is lower than the second flow rate.

8. The method of claim 7, wherein the first flow rate being lower than the second flow rate indicates that fractures have formed in the formation sample.

9. The method of claim 1, wherein the formation sample is a shale sample.

10. The method of claim 1, wherein preparing the testing disk further comprises:
grinding the testing disk.

11. The method of claim 1, further comprising:
fracturing the formation sample.

12. A method for testing and ranking efficacies of sealing and plugging agents, comprising:
preparing a first fluid with a first sealing and plugging agent (SPA);
pressurizing the first fluid at a constant injection pressure and against a first formation sample of a first type;
recording one or more volumes of first fluid effluent over time to determine a first flow rate, wherein the first fluid effluent flows from the first formation sample;
preparing a second fluid with a second SPA;
pressurizing the second fluid at the constant injection pressure and against a second formation sample of the first type;
recording one or more volumes of second fluid effluent over time to determine a second flow rate, wherein the second fluid effluent flows from the second formation sample; and
ranking the first SPA relative to the second SPA based at least in part on the first flow rate and the second flow rate.

13. The method of claim 12, wherein each of the first fluid and the second fluid further comprises a potassium chloride solution.

14. The method of claim 12, wherein each of the first SPA and the second SPA comprises nano-size carbon black.

15. The method of claim 12, wherein the first flow rate being higher than the second flow rate indicates that the first SPA is ranked lower than the second SPA.

16. The method of claim 12, wherein the first type of the first and second formation sample is a shale sample.

17. The method of claim 12, further comprising:
fracturing the first formation sample or the second formation sample.

18. The method of claim 12, wherein first formation sample is assembled in a first testing disk, and wherein the second formation sample is assembled in a second testing disk.

19. The method of claim 18, wherein a portion of each of the first testing disk and the second testing disk further comprises a nonpermeable substance.

20. The method of claim 18, further comprising:
assembling the first testing disk in a Permeability Plugging Apparatus (PPA); and
assembling the second testing disk in the PPA.
